(19) United States
(12) Patent Application Publication
(10) Pub. No.: US 2023/0343466 A1
(43) Pub. Date: Oct. 26, 2023

(54) METHOD AND APPARATUS FOR PROCESSING SLEEPING DATA, COMPUTER DEVICE, PROGRAM AND MEDIUM

(71) Applicant: BOE TECHNOLOGY GROUP CO., LTD., Beijing (CN)

(72) Inventors: Yubin Zhou, Beijing (CN); Jiao Huang, Beijing (CN); Fang Zhai, Beijing (CN); Jianxun Liu, Beijing (CN)

(73) Assignee: BOE TECHNOLOGY GROUP CO., LTD., Beijing (CN)

(21) Appl. No.: 17/635,785

(22) PCT Filed: Apr. 29, 2021
(86) PCT No.: PCT/CN2021/091077

§ 371 Date: Feb. 16, 2022

(51) Int. Cl. G16H 40/67; G16H 50/70; A61B 5/00; G16H 15/00

(52) U.S. Cl. CPC G16H 50/70; A61B 5/4815; G16H 15/00; G16H 40/67

(57) ABSTRACT

Some embodiments of the present disclosure provide a method and an apparatus for processing sleeping data, a computer device, a program and a medium, which relates to the technical field of computers. The method includes: acquiring sleeping data collected by a sleeping monitoring device; extracting a sleeping feature in the sleeping data; performing similarity comparison to a standard feature of a user and the sleeping feature, to obtain a comprehensive feature similarity; and on the condition that the comprehensive feature similarity satisfies a similarity requirement, using the sleeping feature as a target sleeping feature of the user.

### TECHNICAL FIELD

The present disclosure relates to the technical field of computers, and particularly relates to a method and an apparatus for processing sleeping data, a computer device, a program and a medium.

### BACKGROUND

Sleeping monitoring is a method that, by using sleeping monitoring instrument, monitors events during the sleeping of the user that can reflect the sleeping state of the user, such as respiration and heartbeat, and in turn analyzes and processes the data obtained by the monitoring to assess the sleeping state of the user, and facilitates the user to know and improve the sleeping quality.

### SUMMARY

The present disclosure provides a method and an apparatus for processing sleeping data, a computer device, a program and a medium, which aims at solving to the largest extent the problem in the related art that, because the attribution of the sleeping data relies on the binding relation between the sleeping monitoring device and the user, the accuracy of the attribution of the sleeping data is reduced.

Some embodiments of the present disclosure provide a method for processing sleeping data, wherein the method comprises:
acquiring sleeping data collected by a sleeping monitoring device;
extracting a sleeping feature in the sleeping data;
performing similarity comparison to a standard feature of a user and the sleeping feature, to obtain a comprehensive feature similarity; and
on the condition that the comprehensive feature similarity satisfies a similarity requirement, using the sleeping feature as a target sleeping feature of the user.

Optionally, the step of extracting the sleeping feature in the sleeping data comprises:
by using sleeping algorithms corresponding to sleeping sub-stages, acquiring sleeping datasets individually matching with the sleeping sub-stages in the sleeping data, and a sleeping-period time sequence of the sleeping data; and
according to the sleeping-period time sequence and the sleeping datasets, acquiring the sleeping feature.

Optionally, the sleeping feature comprises at least: a respiration feature and a heartbeat feature; and
the step of performing the similarity comparison to the standard feature of the user and the sleeping feature, to obtain the comprehensive feature similarity comprises:
   according to the sleeping-period time sequence, dividing the respiration feature and the heartbeat feature, to obtain sub-stage-feature sets corresponding to the sleeping sub-stages;
   comparing each of the sub-stage-feature sets with the standard feature, to obtain feature similarities corresponding to the sleeping sub-stages; and
   by using weight values corresponding to the sleeping sub-stages, integrating the feature similarities, to obtain the comprehensive feature similarity.

Optionally, before the step of extracting the sleeping feature in the sleeping data, the method further comprises:

filtering data from the sleeping data that satisfy an ineffective-data requirement, wherein the ineffective-data requirement comprises at least one of an ineffective-data-format requirement and an ineffective-data-valuing requirement.

Optionally, the sleeping feature comprises at least: a sleeping quality; and
the step of, according to the sleeping-period time sequence and the sleeping datasets, acquiring the sleeping feature comprises:
   according to the sleeping datasets and the sleeping-period time sequence, acquiring an apnea-hypopnea index, an awakening time quantity, a falling-asleep duration, a sleeping duration and a sleeping efficiency; and
   integrating the apnea-hypopnea index, the awakening time quantity, the falling-asleep duration, the sleeping duration and the sleeping efficiency, to obtain the sleeping quality.

Optionally, the step of acquiring the sleeping data collected by the sleeping monitoring device comprises:
receiving heartbeat messages periodically reported by the sleeping monitoring device;
extracting a device state in the heartbeat messages;
on the condition that the device state is an operating state, sending a data acquiring request to the sleeping monitoring device; and
receiving the sleeping data that are sent by the sleeping monitoring device according to the data acquiring request.

Optionally, before the step of receiving the heartbeat messages periodically reported by the sleeping monitoring device, the method further comprises:

acquiring a current time from a time calibrating server, to perform clock synchronization with the sleeping monitoring device.

Optionally, after the step of using the sleeping feature as the target sleeping feature of the user, the method further comprises:
extracting, from a sleeping-suggestion information base, a target sleeping-suggestion information that matches with the target sleeping feature and a user information, and according to the target sleeping feature, generating a sleeping view; and
sending to a client a sleeping report that is formed by the sleeping view and the target sleeping-suggestion information, so that the client exhibits the sleeping report.

Optionally, before the step of sending to the client the sleeping report that is formed by the sleeping view and the target sleeping-suggestion information, the method further comprises:

combining the sleeping view in a preset time period with the target sleeping-suggestion information, to obtain the sleeping report corresponding to the preset time period.

Optionally, the step of combining the sleeping view in the preset time period with the target sleeping-suggestion information, to obtain the sleeping report corresponding to the preset time period comprises:
according to an operation parameter of the sleeping monitoring device, generating an operation-indicator information; and
combining the sleeping view in the preset time period, the target sleeping-suggestion information and the operation-indicator information, to obtain the sleeping report corresponding to the preset time period.

Optionally, the step of extracting, from the sleeping-suggestion information base, the target sleeping-suggestion information that matches with the target sleeping feature and the user information comprises:
extracting, from the sleeping-suggestion information base, a sleeping-suggestion information that matches with the target sleeping feature and the user information; and
extracting, from the sleeping-suggestion information, the target sleeping-suggestion information that satisfies a user-configuration type, wherein the user-configuration type comprises at least one of an audio type, a video type and a text type.

Some embodiments of the present disclosure further provide an apparatus for processing sleeping data, wherein the apparatus comprises:
a receiving module configured for acquiring sleeping data collected by a sleeping monitoring device;
an extracting module configured for extracting a sleeping feature in the sleeping data;
a comparing module configured for performing similarity comparison to a standard feature of a user and the sleeping feature, to obtain a comprehensive feature similarity; and
a collecting module configured for, on the condition that the comprehensive feature similarity satisfies a similarity requirement, using the sleeping feature as a target sleeping feature of the user.

Optionally, the extracting module is further configured for
by using sleeping algorithms corresponding to sleeping sub-stages, acquiring sleeping datasets individually matching with the sleeping sub-stages in the sleeping data, and a sleeping-period time sequence of the sleeping data; and
according to the sleeping-period time sequence and the sleeping datasets, acquiring the sleeping feature.

Optionally, the sleeping feature comprises at least: a respiration feature and a heartbeat feature; and
the comparing module is further configured for:
   according to the sleeping-period time sequence, dividing the respiration feature and the heartbeat feature, to obtain sub-stage-feature sets corresponding to the sleeping sub-stages;
   comparing each of the sub-stage-feature sets with the standard feature, to obtain feature similarities corresponding to the sleeping sub-stages; and
   by using weight values corresponding to the sleeping sub-stages, integrating the feature similarities, to obtain the comprehensive feature similarity.

Optionally, the extracting module is further configured for:

filtering data from the sleeping data that satisfy an ineffective-data requirement, wherein the ineffective-data requirement comprises at least one of an ineffective-data-format requirement and an ineffective-data-valuing requirement.

Optionally, the sleeping feature comprises at least: a sleeping quality; and
the comparing module is further configured for:
   according to the sleeping datasets and the sleeping-period time sequence, acquiring an apnea-hypopnea index, an awakening time quantity, a falling-asleep duration, a sleeping duration and a sleeping efficiency; and
   integrating the apnea-hypopnea index, the awakening time quantity, the falling-asleep duration, the sleeping duration and the sleeping efficiency, to obtain the sleeping quality.

Optionally, the receiving module is further configured for:
receiving heartbeat messages periodically reported by the sleeping monitoring device;
extracting a device state in the heartbeat messages;
on the condition that the device state is an operating state, sending a data acquiring request to the sleeping monitoring device; and
receiving the sleeping data that are sent by the sleeping monitoring device according to the data acquiring request.

Optionally, the receiving module is further configured for:

acquiring a current time from a time calibrating server, to perform clock synchronization with the sleeping monitoring device.

Optionally, the apparatus further comprises: an outputting module configured for:
extracting, from a sleeping-suggestion information base, a target sleeping-suggestion information that matches with the target sleeping feature and a user information, and according to the target sleeping feature, generating a sleeping view; and
sending to a client a sleeping report that is formed by the sleeping view and the target sleeping-suggestion information, so that the client exhibits the sleeping report.

Optionally, the outputting module is further configured for:

combining the sleeping view in a preset time period with the target sleeping-suggestion information, to obtain a sleeping report corresponding to the preset time period.

Optionally, the outputting module is further configured for:
according to an operation parameter of the sleeping monitoring device, generating an operation-indicator information; and
combining the sleeping view in the preset time period, the target sleeping-suggestion information and the operation-indicator information, to obtain the sleeping report corresponding to the preset time period.

Optionally, the outputting module is further configured for:
extracting, from the sleeping-suggestion information base, a sleeping-suggestion information that matches with the target sleeping feature and the user information; and
extracting, from the sleeping-suggestion information, the target sleeping-suggestion information that satisfies a user-configuration type, wherein the user-configuration type comprises at least one of an audio type, a video type and a text type.

Some embodiments of the present disclosure further provide a computing and processing device, wherein the computing and processing device comprises:
a memory storing a computer-readable code; and
one or more processors, wherein when the computer-readable code is executed by the one or more processors, the computing and processing device implements the method for processing sleeping data stated above.

Some embodiments of the present disclosure further provide a computer program, wherein the computer program comprises a computer-readable code, and when the computer-readable code is executed in a computing and processing device, the computer-readable code causes the computing and processing device to implement the method for processing sleeping data stated above.

Some embodiments of the present disclosure further provide a computer-readable medium, wherein the computer-readable medium stores a computer program of the method for processing sleeping data stated above.

The above description is merely a summary of the technical solutions of the present disclosure. In order to more clearly know the elements of the present disclosure to enable the implementation according to the contents of the description, and in order to make the above and other purposes, features and advantages of the present disclosure more apparent and understandable, the particular embodiments of the present disclosure are provided below.

### BRIEF DESCRIPTION OF THE DRAWINGS

In order to more clearly illustrate the technical solutions of the embodiments of the present disclosure or the prior art, the figures that are required to describe the embodiments or the prior art will be briefly introduced below. Apparently, the figures that are described below are embodiments of the present disclosure, and a person skilled in the art can obtain other figures according to these figures without paying creative work

FIG. 1 schematically shows a schematic flow chart of the method for processing sleeping data according to some embodiments of the present disclosure.

FIG. 2 schematically shows a schematic logic diagram of the firmware updating method of the sleeping monitoring device according to some embodiments of the present disclosure.

FIG. 3 schematically shows a schematic flow chart of another firmware updating method of the sleeping monitoring device according to some embodiments of the present disclosure.

FIG. 4 schematically shows a schematic principle diagram of the sleeping-stage dividing method according to some embodiments of the present disclosure.

FIG. 5 schematically shows a schematic effect diagram of the sleeping view according to some embodiments of the present disclosure.

FIG. 6 schematically shows a schematic flow chart of the method for acquiring a sleeping quality according to some embodiments of the present disclosure.

FIG. 7 schematically shows a schematic flow chart of the method for generating a sleeping report according to some embodiments of the present disclosure.

FIG. 8 schematically shows a schematic flow chart of the method for acquiring a sleeping-suggestion information according to some embodiments of the present disclosure.

FIG. 9 schematically shows a schematic logic diagram of the method for processing sleeping data according to some embodiments of the present disclosure.

FIG. 10 schematically shows a schematic structural diagram of the apparatus for processing sleeping data according to some embodiments of the present disclosure.

FIG. 11 schematically shows a block diagram of a computing and processing device for implementing the method according to the present disclosure.

FIG. 12 schematically shows a storage unit for maintaining or carrying a program code for implementing the method according to the present disclosure.

### DETAILED DESCRIPTION

In order to make the objects, the technical solutions and the advantages of the embodiments of the present disclosure clearer, the technical solutions of the embodiments of the present disclosure will be clearly and completely described below with reference to the drawings of the embodiments of the present disclosure. Apparently, the described embodiments are merely certain embodiments of the present disclosure, rather than all of the embodiments. All of the other embodiments that a person skilled in the art obtains on the basis of the embodiments of the present disclosure without paying creative work fall within the protection scope of the present disclosure.

In the related art, a sleeping monitoring device is generally bound in advance with the user, and in turn attributes the collected sleeping data to the user bound therewith. However, when the same sleeping monitoring device is required to be used by multiple users, in the case in which the binding between the sleeping monitoring device and the user is not changed timely, the sleeping data belonging to one user are attributed to another user, which seriously affects the accuracy of the attribution of the sleeping data.

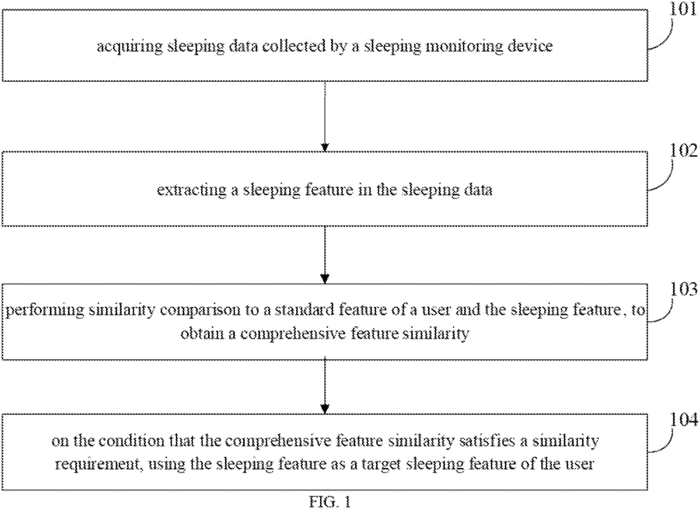

FIG. 1 schematically shows a schematic flow chart of the method for processing sleeping data according to some embodiments of the present disclosure. The method includes:

Step 101: acquiring sleeping data collected by a sleeping monitoring device.

In an embodiment of the present disclosure, the sleeping monitoring device refers to a device that generates a sleeping monitoring signal, and the sleeping monitoring device can access an Internet of Things via a wireless network, to realize the data interaction between the sleeping monitoring device and the service side of the Internet of Things. The sleeping monitoring device may monitor the sleeping action of the user by using a non-contacting radar wave, whereby the user is not required to wear the sleeping monitoring device. Certainly, the sleeping monitoring device may also be wearable, which is applicable to the method for processing sleeping data according to some embodiments of the present disclosure, as long as the sleeping monitoring device can be connected to the service side via a network, which may be particularly configured according to practical demands, and is not limited herein. The sleeping monitoring device, in the operating state, automatically starts up the sleeping monitoring by using preset starting and ending times of the monitoring, and the user may also customize them by using a mobile terminal. The user may configure the sleeping monitoring device by using a mobile terminal, such as the network information, the sleeping pattern and the breathing-light state. The operation state of the sleeping monitoring device is synchronized to the mobile terminal of the user in real time, which facilitates the user to view the parameters of the polysomnography and the relevant preference.

In practical applications, the sleeping monitoring device may monitor the actions of the user that can reflect the sleeping state, such as respiration, heartbeat and body movement, by using a built-in pressure sensor, sonic sensor and so on, to generate continuous original signals of the sleeping monitoring parameters. Subsequently, the sleeping monitoring device may perform digital-analog conversion processing and data assembling to the original information by using a built-in information processing module, to generate formatted data in a particular programming language as the sleeping data. Certainly, the sleeping monitoring device may update the program versions of the functional modules by using a pluggable program firmware, and may also update the program versions by remote interaction with a server. The sleeping monitoring device may also include a locally storing unit, which serves as a temporary database of the sleeping data, and is used for the data storage of the early-stage interaction of the data interaction with the service side. The sleeping monitoring device may also include an interface-transmission requesting module, which serves for the network transmission and the information interaction with the service side, performs data assembling and remote invoking by complying with an agreed interface protocol, and transmits the local-area-network data to the service side. It should be noted that, because the sleeping monitoring device has a limited volume, it has a restricted hardware configuration, and, thus, low storage capacity, data transmission capacity and data processing capability, and a service side that matches with the sleeping monitoring device may be provided to serve for the functions of storage, processing, transmission and so on of the sleeping data. The service side may be communicatively connected to the sleeping monitoring device by transmission modes such as a Bluetooth, a wireless network and a mobile network, to realize real-time data interaction between the sleeping monitoring device and the service side, which prevents the risk of losing of the sleeping data caused by insufficient storage resource of the sleeping monitoring device.

For example, the sleeping monitoring device may send the collected sleeping data to the service side, the service side sends the sleeping data to a remote server, and the remote server completes the further processing on the sleeping data. The remote server may be a distributed server cluster, and, accordingly, after the sleeping data sent by the service side is received, the sleeping data may be processed by a distributed server that is leisure or has a low load according to the distribution scheduling task, which does not only increase the resource utilization ratio of the server, but also can increase the processing efficiency of the sleeping data.

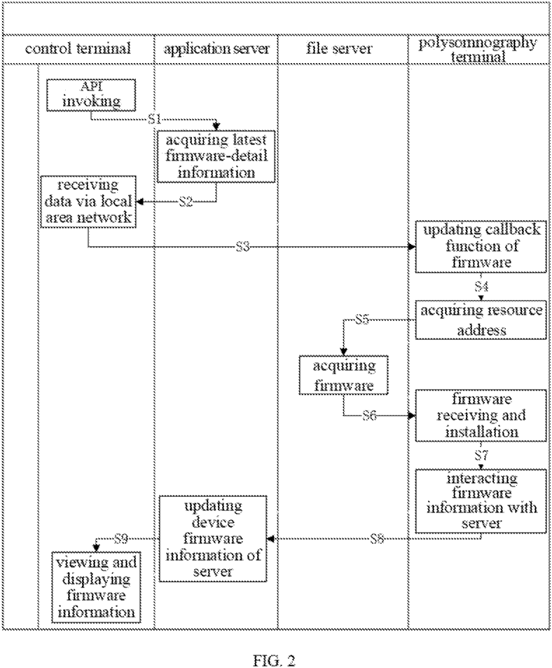

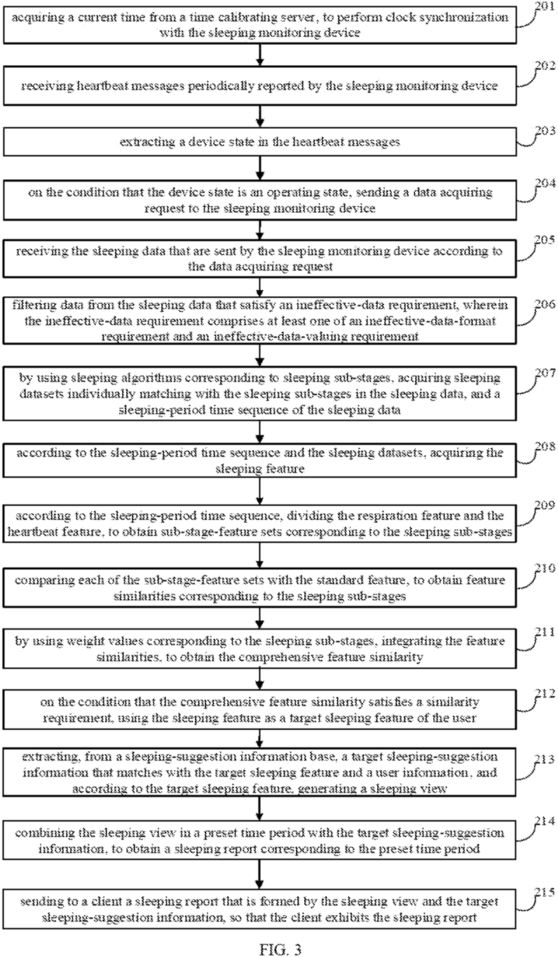

As an example, referring to FIG. 2, the present application further provides a firmware updating method of the sleeping monitoring device. The controlling terminal refers to a terminal that serves for controlling the updating of the firmware. The application server refers to a server that sends a firmware information. The file server refers to a server that stores the firmware information. The polysomnography terminal refers to the sleeping monitoring device. The method includes:
Step S1: by the controlling terminal, invoking an API interface of the application server, to acquire a latest firmware-detail information;
Step S2: by the controlling terminal, receiving the latest firmware-detail information sent by the application server,
Step S3: by the controlling terminal, sending an updating instruction to the polysomnography terminal;
Step S4: by the polysomnography terminal, according to the updating instruction, invoking a firmware updating function to acquire a resource address;
Step S5: by the polysomnography terminal, acquiring a firmware updating packet of the required updating from the file server,
Step S6: by the polysomnography terminal, installing the received firmware updating packet;
Step S7: by the polysomnography terminal, upgrading according to the firmware;
Step S8: by the polysomnography terminal, sending updated firmware information to the application server, so that the application server updates the device firmware information corresponding to the polysomnography terminal; and
Step S9: by the controlling terminal, acquiring updated device firmware information from the application server, and displaying the updated device firmware information.

Certainly, the above is merely illustrative description on the firmware updating method of the sleeping monitoring device, and, particularly, the firmware of the sleeping monitoring device may also be updated by other modes of firmware updating, for example by replacing a pluggable program firmware, and by field updating by maintenance personnel, which may be particularly configured according to practical demands, and is not limited herein.

Step 102: extracting a sleeping feature in the sleeping data.

In an embodiment of the present disclosure, the sleeping feature refers to an indicator parameter that can reflect the sleeping state of the user, for example a sleeping efficiency, a sleeping-quality score and a sleeping duration. The sleeping feature may be raw data that are extracted directly from the sleeping data, and may also be an indicator parameter that is obtained by secondary processing to the sleeping data. It can be understood that, because the sleeping data might contain interfering data that are irrelevant to the sleeping state of the user, for example the talking-sound data and the walking-sound data of the other users that are located in the same room as the user, or the heartbeat data, the respiration data and so on before the user falls asleep, it is required to selectively extract from the sleeping data. Particularly, the specific indicator data in the sleeping data may be identified by using a predetermined sleeping algorithm, and in turn that part of the sleeping data may be extracted as the sleeping feature. For example, the heartbeat data in the sleeping data may be identified by providing a heartbeat identifying algorithm, or the heart-rate data in the sleeping data may be identified by using a heart-rate algorithm, and so on. The particular sleeping feature may be configured by providing different sleeping algorithms according to practical demands, and are not limited herein.

Step 103: performing similarity comparison to a standard feature of a user and the sleeping feature, to obtain a comprehensive feature similarity.

In an embodiment of the present disclosure, the standard feature refers to a feature information that can reflect the sleeping state of a single user, and the standard feature may be obtained by performing feature extraction to the sleeping data of a single user. It can be understood that, because the sleeping monitoring device might be continuously used by multiple users, it is difficult to define which sleeping data in the sleeping data of the users should be attributed to which user, which results in inaccuracy of the attribution of the sleeping data.

In an embodiment of the present disclosure, the sleeping data of each of the users are collected in advance to extract the standard features as reference, and the incidence relation between the user identity informations and the standard features are established and stored. Accordingly, when the user is practically using the sleeping monitoring device, the remote server, according to the identity information of the user, inquires the associated standard feature and performs similarity comparison with the sleeping feature in the sleeping data received this time, to identify which user the sleeping data actually belong to. Particularly, the similarities between each of the standard features and the sleeping feature are calculated, wherein in the calculation the standard features and the features among the sleeping features of the same dimension may be compared individually to obtain the similarities of the features of each of the dimensions, and subsequently the similarities of the features of each of the dimensions may be integrated, whereby a comprehensive feature similarity that can reflect the overall similarity of the features can be obtained.

Step 104: on the condition that the comprehensive feature similarity satisfies a similarity requirement, using the sleeping feature as a target sleeping feature of the user.

In an embodiment of the present disclosure, the similarity requirement refers to the requirement on the value that the comprehensive feature similarity is required to satisfy when the sleeping feature is attributed to the user associated with the standard feature. It may be that the comprehensive feature similarity is greater than a specific similarity threshold, and may also be that the comprehensive feature similarity is within a specific similarity range. Furthermore, the similarity requirement may be pre-set artificially, and may also be automatically configured by a remote server based on the user information. For example, when the quantity of the users associated with the standard feature is large, a larger similarity threshold may be set, while when the quantity of the users associated with the standard feature is small, a smaller similarity threshold may be set. Certainly, the similarity requirement may be configured particularly according to practical demands, which is not limited herein.

In practical situations, if the comprehensive feature similarity satisfies the similarity requirement, then it can be confirmed that the user sleeping state reflected by the sleeping feature is consistent with the standard feature, and therefore the sleeping feature may be attributed to the target sleeping feature of the user associated with the standard feature.

In the embodiments of the present disclosure, by comparing the sleeping feature in the sleeping data collected by the sleeping monitoring device with the standard feature of the user, only when the comprehensive similarity of the comparison between them satisfies the similarity requirement, the sleeping feature is attributed to the user, and the attributed user of the sleeping data can be accurately determined without relying on the binding relation between the sleeping monitoring device and the user.

FIG. 3 schematically shows a schematic flow chart of another method for processing sleeping data according to some embodiments of the present disclosure. The method includes:

Step 201: acquiring a current time from a time calibrating server, to perform clock synchronization with the sleeping monitoring device.

In an embodiment of the present disclosure, the time calibrating server (NTP, Network Time Protocol) refers to a server that is used to provide a high-precision time information to provide a time rectifying function for the connected device. The remote server and the service side connected to the sleeping monitoring device may be connected to the time calibrating server, whereby they can periodically perform information interaction with the time calibrating server, to calibrate the local current time by using the standard time provided by the time calibrating server, thereby ensuring the time synchronization between the sleeping monitoring device and the remote server, and preventing data-transmission delay caused by time error.

Step 202: receiving heartbeat messages periodically reported by the sleeping monitoring device.

In an embodiment of the present disclosure, the heartbeat message refers to a datagram that can reflect the operation state of the sleeping monitoring device. The heartbeat message may contain: device-configuration information such as the device operation state, the network information, the sleeping pattern, the monitoring duration, the sleeping aiding mode, the smart awakening and the report playing.

The sleeping monitoring device periodically and actively sends the heartbeat message to the remote server, and the remote server, in response to the heartbeat message, performs device checking and receives the sleeping data, performs format conversion to the sleeping data to realize the standardization processing to the sleeping data, and stores in a database for subsequent processing. Certainly, the service side connected to the sleeping monitoring device may also, according to the operation state, the network information and so on in the heartbeat message, send to, for example, an application client in the mobile phone of the user for displaying, so that the user can view the operation state of the sleeping monitoring device in real time.

Step 203: extracting a device state in the heartbeat messages.

In an embodiment of the present disclosure, the device state refers to the operation state of the sleeping monitoring device. The device state may be an operating state, a standby state, a shut-down state and so on, which may be particularly configured according to practical demands, and is not limited herein.

Step 204: on the condition that the device state is an operating state, sending a data acquiring request to the sleeping monitoring device.

In an embodiment of the present disclosure, the remote server, when the device state in the heartbeat message is monitored as the operating state, actively sends the data acquiring request to the service side connected to the sleeping monitoring device, so as to timely acquire the sleeping data collected by the sleeping monitoring device.

Step 205: receiving the sleeping data that are sent by the sleeping monitoring device according to the data acquiring request.

In an embodiment of the present disclosure, the service side connected to the sleeping monitoring device, after the data acquiring request sent by the remote server is monitored, extracts the sleeping data from a temporary storage module and sends to the remote server, and after the sending is completed, the service side may delete the sleeping data that is sent, to ensure a rich local storage resource. Particularly, the service side of the sleeping monitoring device may, by invoking the API (Application Programming Interface) of the remote server, send the sleeping data to the remote server.

In the embodiments of the present disclosure, by periodically performing the heartbeat-message interaction between the sleeping monitoring device and the remote server to determine whether the current network transmission link is unobstructed, it is ensured that the sleeping data collected by the sleeping monitoring device can be sent to the remote server timely, which prevents the hidden danger of data losing caused by delayed data transmission.

Step 206: filtering data from the sleeping data that satisfy an ineffective-data requirement, wherein the ineffective-data requirement includes at least one of an ineffective-data-format requirement and an ineffective-data-valuing requirement.

In an embodiment of the present disclosure, the ineffective-data requirement refers to an requirement that the data that cannot reflect the true sleeping state of the user or will affect the sleeping-state analysis satisfy. It can be understood that, when the sleeping monitoring device is performing the sleeping monitoring, because of the interference by irrelevant external factors, some irrelevant data are collected, or, in the transmission of the sleeping data, part of the data are broken, and thus are no longer usable. However, those ineffective data have the characteristics of specific data formats and data values, and therefore the remote server may filter the ineffective data in the sleeping data by providing an ineffective-data-format requirement and an ineffective-data-format requirement, thereby preventing the interference by the ineffective data on the subsequent data processing, and increasing the accuracy of the obtained sleeping feature.

Step 207: by using sleeping algorithms corresponding to sleeping sub-stages, acquiring sleeping datasets individually matching with the sleeping sub-stages in the sleeping data, and a sleeping-period time sequence of the sleeping data.

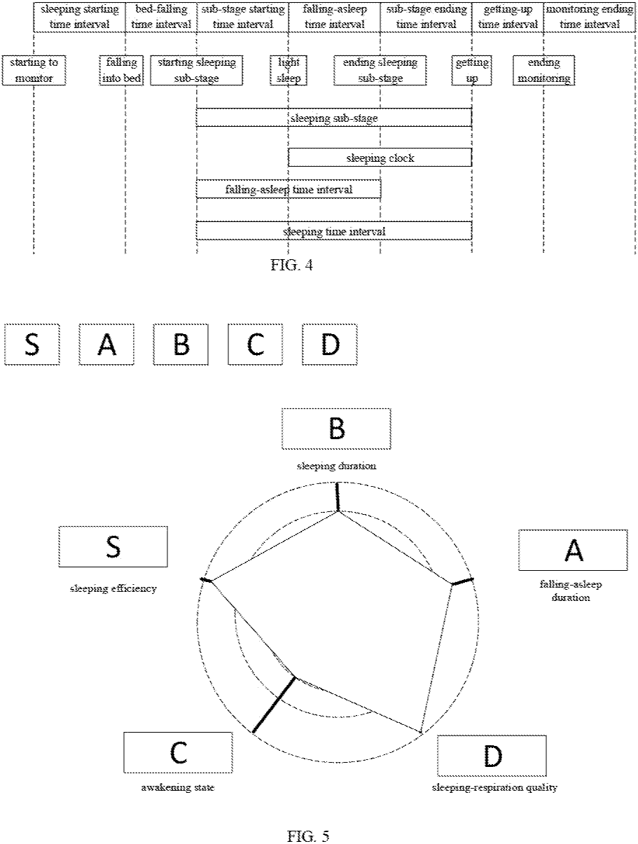

In an embodiment of the present disclosure, the sleeping sub-stages refer to the duration sub-stages when the user is in different states in the sleeping period. For example, referring to FIG. 4, the whole sleeping period may be divided into a sleeping starting time interval, a bed-falling time interval, a sub-stage starting time interval, a falling-asleep time interval, a sub-stage ending time interval, a getting-up time interval and a monitoring ending time interval. The sleeping monitoring on the user is started at the starting moment of the sleeping starting time interval, the user starts to fall into bed at the starting moment of the bed-falling time interval, the sleeping sub-stage is started at the starting moment of the sub-stage starting time interval, the user starts the light sleep at the starting moment of the falling-asleep time interval, the sleeping sub-stage is ended at the starting moment of the sub-stage ending time interval, the user starts to get up at the starting moment of the getting-up time interval, and the sleeping monitoring on the user is ended at the ending moment of the getting-up time interval The sleeping sub-stage is relative to the time intervals before and after the sleeping of the user, and therefore the sleeping sub-stage may be equal to the ending moment of the sub-stage ending time interval subtracting the ending moment of the sub-stage starting time interval. The sleeping clock is a clock for timing the sleeping process of the user, and therefore the sleeping clock may be equal to the sleeping starting time interval plus the sub-stage ending time interval. The falling-asleep time interval refers to the time interval from the soberness to falling asleep of the user, and therefore the falling-asleep time interval may be equal to the starting moment of the falling-asleep time interval subtracting the starting moment of the sub-stage starting time interval. The sleeping time interval refers to the time interval from soberness to falling asleep to awakening of the user, and therefore the sleeping time interval may be equal to the ending moment of the sub-stage ending time interval subtracting the starting moment of the sub-stage starting time interval

The falling-asleep sub-stage refers to the time period from the user lying to bed and being sober to falling asleep, the light-sleep sub-stage refers to the time period when the user is in light sleep, the deep-sleep sub-stage refers to the time period when the user is in deep sleep, and so on. The above are merely exemplary illustrations, and the particular mode of dividing the sleeping sub-stage may be configured according to practical demands, and is not limited herein.

Particularly, the sleeping algorithms corresponding to the different sleeping sub-stages may be configured to identify the sleeping data in the different sleeping sub-stages, to in tum acquire a sleeping-period time sequence that can reflect the time periods that the different sleeping sub-stages are within. For example, by performing data ordering to a whole sleeping period by minutes, a sleeping-period time sequence such as [1,3,3,3,3,3,3,3,2,2,2,2,2,3,3,3,3,3,3,3,3,3,3,3,4,4,4,4,4,3,3,3...] can be obtained, wherein 1 is a soberness period, 2 is an eye-movement period, 3 is a light-sleep period, 4 is a deep-sleep period and 5 is an ineffectiveness period.

Optionally, the sleeping data contained in the different sleeping sub-stages may be collected according to the sleeping-period time sequence, and, accordingly, the sleeping datasets in the sleeping sub-stages can be obtained. For example, the sleeping data of the times of the 1, 2, 3, 4 and 5 in the above-described sleeping-period time sequence may be individually collected as one sleeping dataset, to obtain 5 sleeping datasets that match with the 5 sleeping sub-stages.

Step 208: according to the sleeping-period time sequence and the sleeping datasets, acquiring the sleeping feature.

In an embodiment of the present disclosure, the proportions and the time points of the different sleeping sub-stages in the whole sleeping period may be determined according to the sleeping-period time sequence, and the sleeping datasets may provide the sleeping data in the sleeping sub-stages. By using those data, by calculating by using the algorithms of various sleeping indicators or directly providing the sleeping data in a specific sleeping sub-stage, the sleeping feature that can reflect the sleeping state of the user can be obtained.

Step 209: according to the sleeping-period time sequence, dividing the respiration featme and the heartbeat feature, to obtain sub-stage-feature sets corresponding to the sleeping sub-stages.

In an embodiment of the present disclosure, the respiration feature refers to data that can reflect the respiratory rate of the user, and the heartbeat feature refers to a feature that can reflect the heartbeat frequency of the user. The remote server extracts the respiration feature and the heartbeat feature in the sleeping data, and combines the heartbeat features and the respiration features of each of the sleeping sub-stages, to obtain the sub-stage-feature sets corresponding to the sleeping sub-stages. For example, for the heartbeat feature (heartRateList) and the respiration feature (breathRateList) of the whole monitoring period, according to the time sequence in the sleeping-period time sequence, the sleeping sub-stages that they belong to are found, and are placed into the corresponding sleeping-sub-stage lists, to generate the corresponding sub-stage datasetsheartRateWakeList[], heartRateEyeList[], heartRateLightList[], heartRateDeepList[], heartRateOffList[], breathRateWakeList[], breathRateEyeList[], breathRateLightList[], breathRateDeepList[], and breathRateOffList[].

Step 210: comparing each of the sub-stage-feature sets with the standard feature, to obtain feature similarities corresponding to the sleeping sub-stages.

In an embodiment of the present disclosure, correspondingly to that in the step 209 multiple sub-stage-feature sets may exist, the standard feature may be multiple standard features, which correspond to the different sleeping sub-stages. Therefore, by comparing the standard features of the sleeping datasets corresponding to the different sleeping sub-stages, the feature similarities corresponding to the sleeping sub-stages can be obtained, wherein the modes of calculating the feature similarities may refer to the modes of calculating similarities in the related art, and is not discussed herein further.

Step 211: by using weight values corresponding to the sleeping sub-stages, integrating the feature similarities, to obtain the comprehensive feature similarity.

In an embodiment of the present application, the weight values corresponding to the sleeping sub-stages are set in advance. The weight values may be set by referring to the contributions on the user sleeping state of each of the sleeping sub-stages, and may also be set as average, which may be particularly determined according to practical demands, and is not limited herein.

By performing weighted summation to the feature similarities corresponding to the sleeping sub-stages, the comprehensive feature similarity that can reflect the whole sleeping period can be obtained.

The weight values of the five sleeping sub-stages of the soberness period w1, the eye-movement period w2, the light-sleep period w3, the deep-sleep period w4 and the ineffectiveness period w5 are set, and then the comprehensive feature similarity is calculated by using the following formula (1):

$$\text{sim=} \sum {\text{p}}_{i} {w}_{i} \qquad \text{(1)}$$

wherein sim is the comprehensive feature similarity, pᵢ is the i-th sub-stage dataset, and wᵢ is the weight value of the i-th sub-stage dataset.

Step 212: in response to the comprehensive feature similarity satisfying a similarity requirement, using the sleeping feature as a target sleeping feature of the user.

This step may refer to the detailed description on the step 104, and is not discussed herein further.

Step 213: extracting, from a sleeping-suggestion information base, a target sleeping-suggestion information that matches with the target sleeping feature and a user information, and according to the target sleeping feature, generating a sleeping view.

In an embodiment of the present disclosure, the sleeping-suggestion information base stores the incidence relation between different target sleeping features and sleeping-suggestion informations. The sleeping-suggestion information refers to information that is in advance specified by using practical experience for the sleeping improvement of users of different sleeping features, and may be a sleeping improvement lesson video, a sleeping improvement information and so on. The form of the target sleeping-suggestion information may be configured according to practical demands, and is not limited herein. The sleeping view is obtained by visualization processing to the indicator data in various dimensions in the target sleeping feature, for example a dimension polygon diagram, i.e., setting the quantity of the corners of the polygon according to the dimensions of the indicator data, and representing the numerical values of the indicator data by using the distances from the vertexes of the corners to the center of the polygon. Certainly, it may also be a radar map, a histogram, a sector diagram and a scatter diagram. For example, referring to FIG. 5, wherein S represents the sleeping efficiency, A represents the falling-asleep duration, B represents the sleeping duration, C represents the awakening state, and D represents the sleeping-respiration quality, and a five-dimensional radar map is generated according to the five sleeping features. In it, if the area of the dash area adjacent to the vertex of a certain dimension is higher, then that indicates that the numerical value of the indicator of the sleeping feature corresponding to the vertex of the dimension is higher. Certainly, the above are merely exemplary descriptions, and it is merely required that the user can intuitively know his own sleeping state by using the sleeping view, which is not limited herein.

Step 214: combining a sleeping view in a preset time period with the target sleeping-suggestion information, to obtain a sleeping report corresponding to the preset time period.

In an embodiment of the present disclosure, the preset time period may be per day, per week, per mouth and so on. Accordingly, the obtained sleeping view and target sleeping-suggestion information may be combined according to a predetermined panel template, whereby a sleeping report that can comprehensively reflect the sleeping state of the user can be obtained, such as a simple data report, a daily sleeping report, a weekly sleeping report and a monthly sleeping report.

Step 215: sending to a client a sleeping report that is formed by the sleeping view and the target sleeping-suggestion information, so that the client exhibits the sleeping report.

In an embodiment of the present disclosure, the remote server may send the sleeping report to a client in a terminal device of the user such as a mobile phone, a tablet personal computer and a smart watch, whereby the user can conveniently see the sleeping report by using the client to know his own sleeping state.

Optionally, the sleeping feature includes at least: a sleeping quality.

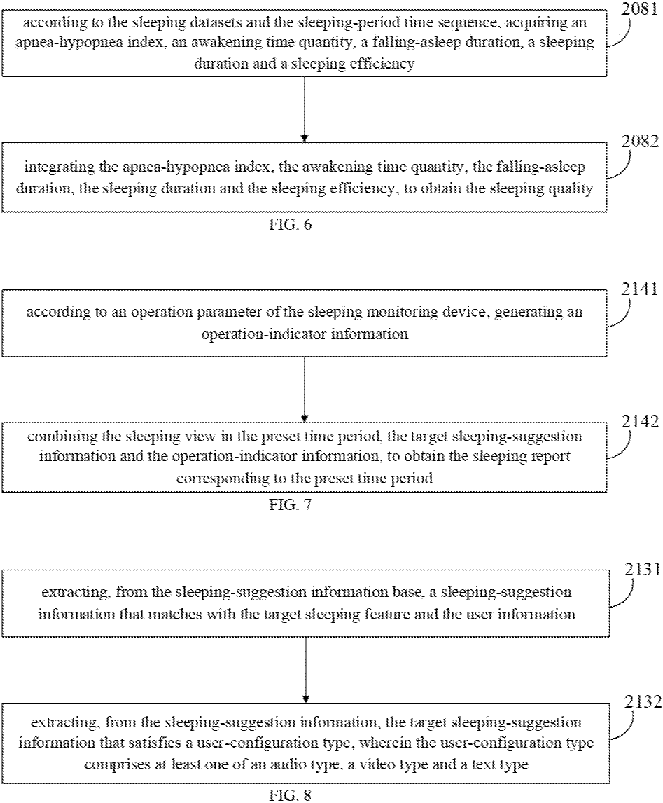

Referring to FIG. 6, the step 208 includes:

Sub-step 2081: according to the sleeping datasets and the sleeping-period time sequence, acquiring an apnea-hypopnea index, an awakening time quantity, a falling-asleep duration, a sleeping duration and a sleeping efficiency.

In an embodiment of the present disclosure, the apnea-hypopnea index (AHI) refers to an index of sleeping apnea and hypopnea per hour of the user. The awakening time quantity refers to, by determining a complying frequency of the soberness periods within the point set from the first deep-sleep period to the last one deep-sleep period within a sleeping period, the quantity of the intervals of the soberness periods of the sleeping-sub-stage diagram that are finally obtained. The falling-asleep duration refers to the duration from the starting of the sleeping sub-stage to the first light sleep. The sleeping efficiency refers to the ratio of the difference between the sleeping duration and the falling-asleep duration to the in-bed duration of the user.

Optionally, the sleeping dataset may further contain the following contents:

The sleep respiration may contain a sleep-respiration-quality index, a low-quality- respiration time quantity, an average low-quality-respiration duration and a longest low-quality-respiration duration. The respiration state of a whole sleeping period is expressed as a two-dimensional array such as [[4572,16,95279,95631],[4571,15,97049,97369],[4701,17,99708,100065]], the first digit XI and the second digit X2 of the inner-layer array are used as the apnea and the low-qualify-respiration duration, the quantity of the X1 in the two-dimensional array that are not 0 is the apnea time quantity N1, the quantity of the X2 in the two-dimensional array that are not 0 is the low-quality-respiration time quantity N2, SUM(N1,N2) is the sleep-respiration-quality index, -1 is set to be the ineffective state, MAX(X2) is the longest low-quality-respiration duration, and AVER(X2) is the average low-quality-respiration duration.

The deep-sleep duration refers to a duration of the state in the deep-sleep period within the whole sleeping period in the calculation logic of the sleeping sub-stage. The real-time heart rate and the real-time respiratory rate refer to the real-time heart rate and respiration rate of the user that are acquired by the sleeping monitoring by the sleeping monitoring device, and are placed into a heart-rate-data list heartRateList and a respiratory-rate-data list breathRateList respectively with minute as the unit and in the time sequence. The body movement refers to the body-movement feature of the user acquired by the sleeping monitoring by the sleeping monitoring device, and is placed into a body-movement-data list with minute as the unit and in the time sequence, for example, [0.0,1.0,2.0.1.0], wherein 0.0 represents no movement, 1.0 represents a slight movement and 2.0 represents a dramatic movement. The snore-somniloquy file is stored locally in the polysomnography, and is stored and expressed by the remote server in the form of {“snore™[“Sleep-1571760959-26”],“somniloquy™”:[“Sleep-1571771248-3”]}, wherein snore represents the snore file list, and somniloquy represents the somniloquy file list. If it is required to play the snore and the somniloquy, then the files are acquired locally at the sleeping monitoring device by using the file lists returned via the interface for exhibition and playing.

Sub-step 2082: integrating the apnea-hypopnea index, the awakening time quantity, the falling-asleep duration, the sleeping duration and the sleeping efficiency, to obtain the sleeping quality.

In an embodiment of the present disclosure, firstly, the values of the factors f() of the apnea-hypopnea index (AHI), the awakening time quantity (wakeN), the falling-asleep duration (T1), the sleeping duration (T2) and the sleeping efficiency (X) are calculated, which may be calculated particularly by using the following formmlas (2)-(6):

$$f \left(A H I\right) = \left\{\begin{matrix}0 , A H I < 0 \\ 1 , 5 \leq A H I < 15 \\ 2 , 15 \leq A H I < 30 \\ 3 , A H I \geq 30\end{matrix}\right) \qquad \text{(2)}$$

$$f \left(w a k e N\right) = \left\{\begin{matrix}0 , w a k e N = 0 \\ 1 , w a k e N = 1 \\ 2 , w a k e N = 2 \\ 3 , w a k e N \geq 3\end{matrix}\right) \qquad \text{(3)}$$

$$f \left(T 1\right) = \left\{\begin{matrix}0 , T 1 < 0 \\ 1 , 15 \leq T 1 < 15 \\ 2 , 30 \leq T 1 < 30 \\ 3 , T 1 \geq 30\end{matrix}\right) \qquad \text{(4)}$$

$$f \left(T 2\right) = \left\{\begin{matrix}3 , T 2 < 5 \\ 2 , 5 \leq T 2 < 6 \\ 1 , 6 \leq T 2 < 7 \\ 0 , T 2 \geq 7\end{matrix}\right) \qquad \text{(5)}$$

$$f \left(X\right) = \left\{\begin{matrix}3 , X < 65 \% \\ 2 , 65 \% \leq X < 75 \% \\ 1 , 75 \% \leq X < 85 \% \\ 0 , X \geq 85 \%\end{matrix}\right) \qquad \text{(6)}$$

Subsequently, the values of the factors are integrated by using the following formula (7):

$$\text{Y=} f \left(A H I\right) \text{+} f \left(w a k e N\right) \text{+} f \left(T 1\right) \text{+} f \left(T 2\right) \text{+} f \left(X\right) \qquad \text{(7)}$$

wherein Y is a comprehensive factor value.

Finally, the comprehensive factor value is inputted into the following formula (8) to obtain the sleeping quality of the user:

$$S Q I = \left\{\begin{matrix}99 - 4.5 ∗ \text{Y,} Y ∈ \left(0 ∼ 2\right) \\ 89 - \left(19 / 4\right) ∗ \left(\text{Y} - 3\right) , Y ∈ \left(3 ∼ 7\right) \\ 69 - \left(19 / 4\right) ∗ \left(\text{Y} - 8\right) , Y ∈ \left(8 ∼ 12\right) \\ 49 - 4.5 ∗ \left(\text{Y} - 13\right) , Y ∈ \left(13 ∼ 15\right)\end{matrix}\right) \qquad \text{(8)}$$

wherein SQI is the sleeping quality.

Optionally, referring to FIG. 7, the step 214 includes:

Sub-step 2141: according to an operation parameter of the sleeping monitoring device, generating an operation-indicator information.

In an embodiment of the present disclosure, the operation parameter may be extracted from the heartbeat message that is sent by the service side to the remote server and is provided by the sleeping monitoring device. The operation parameter may reflect the operation mode, the abnormalities and so on during the operation of the sleeping monitoring device. By performing visualization processing to the operation parameter, the operation-indicator information that can reflect the operation state of the sleeping monitoring device can be obtained. For example, the range of the values of a specific parameter among the operation parameters may be monitored, and if they exceed a certain range, a warning information may be generated as the operation-indicator information. Alternatively, according to the operation state, a corresponding icon is generated as the operation-indicator information.

Sub-step 2142: combining the sleeping view in the preset time period, the target sleeping-suggestion information and the operation-indicator information, to obtain the sleeping report corresponding to the preset time period.

In an embodiment of the present disclosure, the sleeping report provided to the user may also contain the operation-indicator information of the sleeping monitoring device within a specific time period, whereby the user can conveniently know the operation state of the sleeping monitoring device by using the sleeping report.

Optionally, referring to FIG. 8, the step 213 includes:

Sub-step 2131: extracting, from the sleeping-suggestion information base, a sleeping-suggestion information that matches with the target sleeping feature and the user information.

In an embodiment of the present disclosure, the sleeping-suggestion information stored in the sleeping-suggestion information base may establish an incidence relation with the sleeping feature and the user information. The user information may be personal information such as the user gender, the user age and the user career, so that sleeping-suggestion informations associated with the different sleeping features may be provided according to the different user informations, to realize customized sleeping suggestions adapted for the user informations, whereby the provided sleeping-suggestion information is more suitable for the actual situations of the user.

Sub-step 2132: extracting, from the sleeping-suggestion information, a target sleeping-suggestion information that satisfies a user-configuration type, wherein the user-configuration type includes at least one of an audio type, a video type and a text type.

FIG. 9 schematically shows a schematic logic diagram of the method for processing sleeping data according to some embodiments of the present disclosure. The method includes:
by a polysomnography device terminal, performing non-contacting sleeping monitoring to the user to collect the sleeping data;
by the polysomnography device terminal, by SmartConfig (one-key network-configuring mode), by using a protocol of the Internet of Things, performing data transmission;
by the polysomnography device terminal, interacting with a remote distributed-application interaction server, to send the operation state, the real-time data and the heartbeat message to the remote distributed-application interaction server, and, by the remote distributed-application interaction server, performing distributed data storage to the operation state, the real-time data and the heartbeat message;
by the polysomnography device terminal, when performing sleeping monitoring to the user, firstly collecting an original signal value of a sleeping monitoring parameter, subsequently obtaining the standard-formatted sleeping data by digital-analog conversion processing and data assembling, subsequently storing the sleeping data to a local terminal database for temporary storage, and finally performing distributed data storage to the sleeping data by using the interface-transmission requesting module;
by the remote distributed-application interaction server, passing the stored sleeping data through a data processing module, passing the sleeping data sequentially through an indicator-generation predetermined-processing-algorithm processing, a sleeping-sub-stage judging processing and a logic-time-sequence processing, and passing to a data collecting module;
by the data collecting module of the remote distributed-application interaction server, extracting the sleeping features in the required sleeping scene from the sleeping data, and collecting the sleeping features by using boundary similarity calculation, to determine the attributed user of the sleeping features; and
by the remote distributed-application interaction server, extracting the sleeping monitoring indicator in the sleeping feature and inquiring the comprehensive improvement suggestion and the sleeping quality assessment that match with the sleeping feature, and subsequently performing data pushing of the sleeping monitoring indicator, the comprehensive improvement suggestion and the sleeping quality assessment, so that the user can view them by using the client.

In an embodiment of the present disclosure, the user-configuration type refers to the type of the required sleeping-suggestion information that is configured by the user. The user-configuration type may be an audio type, a video type, an audio-video type, a text type and so on. For example, information that facilitates to improve the sleeping quality of the user such as a relevant information, an on-line lesson and a sleeping improving service may also be recommended to the user according to the user-configuration type. Certainly, the above are merely exemplary illustrations, which may be particularly configured according to practical demands, and is not limited herein.

The embodiments of the present disclosure are used to recommend customized sleeping-suggestion information to the user according to the user information and the user settings, whereby the sleeping-suggestion information acquired by the user is more suitable for the actual situations of the user, which increases the accuracy of the recommendation of the sleeping-suggestion information.

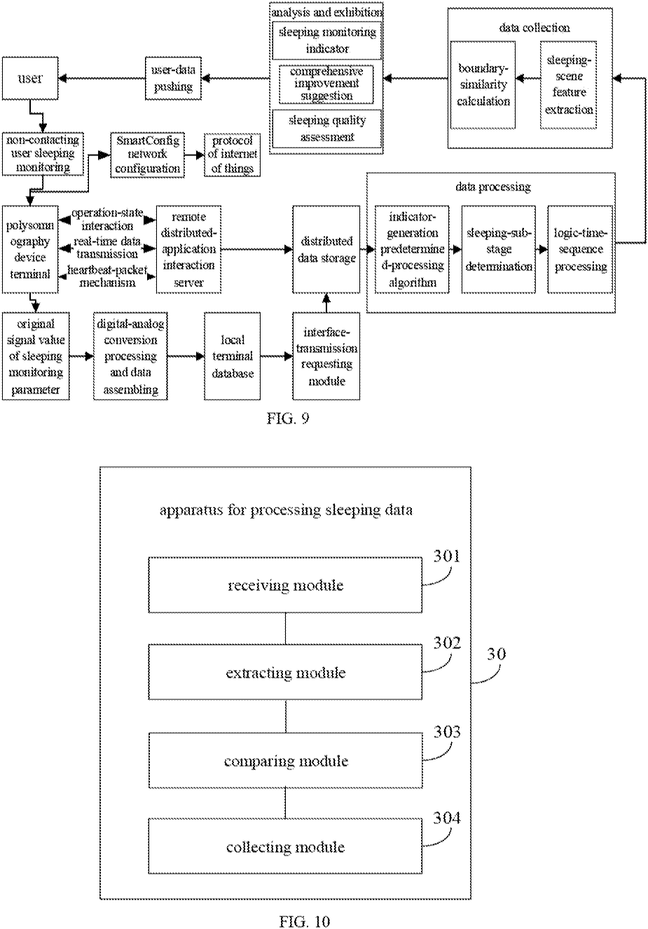

FIG. 10 schematically shows a schematic structural diagram of the apparatus 30 for processing sleeping data according to some embodiments of the present disclosure. The apparatus includes:
a receiving module 301 configured for acquiring sleeping data collected by a sleeping monitoring device;
an extracting module 302 configured for extracting a sleeping feature in the sleeping data;
a comparing module 303 configured for performing similarity comparison to a standard feature of a user and the sleeping feature, to obtain a comprehensive feature similarity; and
a collecting module 304 configured for, on the condition that the comprehensive feature similarity satisfies a similarity requirement, using the sleeping feature as a target sleeping feature of the user.

Optionally, the extracting module 302 is further configured for:
by using sleeping algorithms corresponding to sleeping sub-stages, acquiring sleeping datasets individually matching with the sleeping sub-stages in the sleeping data, and a sleeping-period time sequence of the sleeping data; and
according to the sleeping-period time sequence and the sleeping datasets, acquiring the sleeping feature.

Optionally, the sleeping feature includes at least: a respiration feature and a heartbeat feature; and
the comparing module 303 is further configured for:
   according to the sleeping-period time sequence, dividing the respiration feature and the heartbeat feature, to obtain sub-stage-feature sets corresponding to the sleeping sub-stages;
   comparing each of the sub-stage-feature sets with the standard feature, to obtain feature similarities corresponding to the sleeping sub-stages; and
   by using weight values corresponding to the sleeping sub-stages, integrating the feature similarities, to obtain the comprehensive feature similarity.

Optionally, the extracting module 302 is further configured for:

filtering data from the sleeping data that satisfy an ineffective-data requirement, wherein the ineffective-data requirement includes at least one of an ineffective-data-format requirement and an ineffective-data-valuing requirement.

Optionally, the sleeping feature includes at least: a sleeping quality; and
the comparing module 303 is further configured for:
   according to the sleeping datasets and the sleeping-period time sequence, acquiring an apnea-hypopnea index, an awakening time quantity, a falling-asleep duration, a sleeping duration and a sleeping efficiency; and
   integrating the apnea-hypopnea index, the awakening time quantity, the falling-asleep duration, the sleeping duration and the sleeping efficiency, to obtain the sleeping quality.

Optionally, the receiving module 301 is further configured for:
receiving heartbeat messages periodically reported by the sleeping monitoring device;
extracting a device state in the heartbeat messages;
in response to the device state being an operating state, sending a data acquiring request to the sleeping monitoring device; and
receiving the sleeping data that are sent by the sleeping monitoring device according to the data acquiring request.

Optionally, the receiving module 301 is further configured for:

acquiring a current time from a time calibrating server, to perform clock synchronization with the sleeping monitoring device.

Optionally, the apparatus further includes: an outputting module configured for:
extracting, from a sleeping-suggestion information base, a target sleeping-suggestion information that matches with the target sleeping feature and a user information, and according to the target sleeping feature, generating a sleeping view; and
sending to a client a sleeping report that is formed by the sleeping view and the target sleeping-suggestion information, so that the client exhibits the sleeping report.

Optionally, the outputting module is further configured for:

combining a sleeping view in a preset time period with the target sleeping-suggestion information, to obtain a sleeping report corresponding to the preset time period.

Optionally, the outputting module is further configured for:
according to an operation parameter of the sleeping monitoring device, generating an operation-indicator information; and
combining the sleeping view in the preset time period, the target sleeping-suggestion information and the operation-indicator information, to obtain the sleeping report corresponding to the preset time period.

Optionally, the outputting module is further configured for:
extracting, from the sleeping-suggestion information base, a sleeping-suggestion information that matches with the target sleeping feature and the user information; and
extracting, from the sleeping-suggestion information, a target sleeping-suggestion information that satisfies a user-configuration type, wherein the user-configuration type includes at least one of an audio type, a video type and a text type.

The above-described device embodiments are merely illustrative, wherein the units that are described as separate components may or may not be physically separate, and the components that are displayed as units may or may not be physical units; in other words, they may be located at the same location, and may also be distributed to a plurality of network units. Some or all of the modules may be selected according to the actual demands to realize the purposes of the solutions of the embodiments. A person skilled in the art can understand and implement the technical solutions without paying creative work.

Each component embodiment of the present disclosure may be implemented by hardware, or by software modules that are operated on one or more processors, or by a combination thereof. A person skilled in the art should understand that some or all of the functions of some or all of the components of the computing and processing device according to the embodiments of the present disclosure may be implemented by using a microprocessor or a digital signal processor (DSP) in practice. The present disclosure may also be implemented as apparatus or device programs (for example, computer program and computer program products) for implementing part of or the whole of the method described herein. Such programs for implementing the present disclosure may be stored in a computer-readable medium, or may be in the form of one or more signals. Such signals may be downloaded from an Internet website, or provided on a carrier signal, or provided in any other forms.

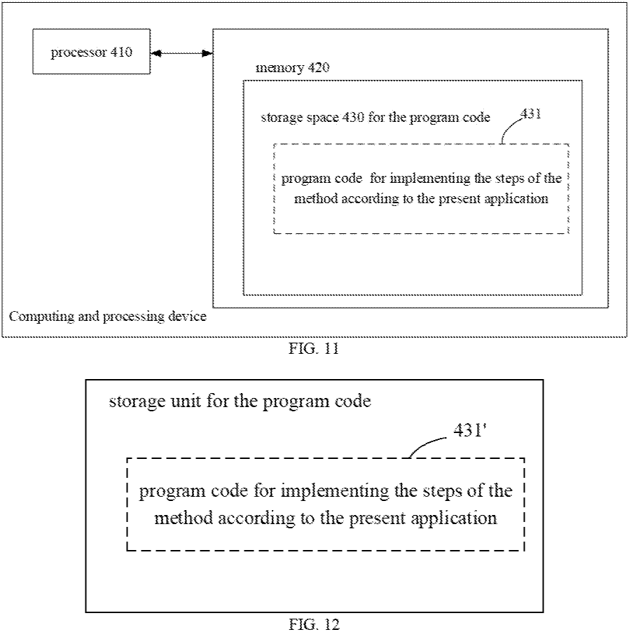

For example, FIG. 11 shows a computing and processing device that can implement the method according to the present disclosure. The computing and processing device traditionally includes a processor 410 and a computer program product or computer-readable medium in the form of a memory 420. The memory 420 may be electronic memories such as flash memory, EEPROM (Electrically Erasable Programmable Read Only Memory), EPROM, hard disk or ROM. The memory 420 has the storage space 430 of the program code 431 for implementing any steps of the above method. For example, the storage space 430 for program code may contain program codes 431 for individually implementing each of the steps of the above method. Those program codes may be read from one or more computer program products or be written into the one or more computer program products. Those computer program products include program code carriers such as a hard disk, a compact disk (CD), a memory card or a floppy disk. Such computer program products are usually portable or fixed storage units as shown in FIG. 12. The storage unit may have storage segments or storage spaces with similar arrangement to the memory 420 of the computing and processing device in FIG. 11. The program codes may, for example, be compressed in a suitable form. Generally, the storage unit contains a computer-readable code 431′, which can be read by a processor like 410. When those codes are executed by the computing and processing device, the codes cause the computing and processing device to implement each of the steps of the method described above.

It should be understood that, although the steps in the flow charts in the drawings are shown sequentially according to the indication by the arrows, those steps are not necessarily performed sequentially according to the sequence indicated by the arrows. Unless expressly described herein, the sequence of the performances of those steps are not strictly limited, and they may be performed in other sequences. Furthermore, at least some of the steps in the flow charts in the drawings may include a plurality of sub-steps or a plurality of stages, wherein those sub-steps or stages are not necessarily completely performed at the same moment, but may be performed at different moments, and their performance sequence is not necessarily sequential performance, but may be performance alternate with at least some of the other steps or the sub-steps or stages of the other steps.

The “one embodiment”, “an embodiment” or “one or more embodiments” as used herein means that particular features, structures or characteristics described with reference to an embodiment are included in at least one embodiment of the present disclosure. Moreover, it should be noted that here an example using the wording “in an embodiment” does not necessarily refer to the same embodiment.

The description provided herein describes many concrete details. However, it can be understood that the embodiments of the present disclosure may be implemented without those concrete details. In some of the embodiments, well-known processes, structures and techniques are not described in detail, so as not to affect the understanding of the description.

In the claims, any reference signs between parentheses should not be construed as limiting the claims. The word “comprise” does not exclude elements or steps that are not listed in the claims. The word “a” or “an” preceding an element does not exclude the existing of a plurality of such elements. The present disclosure may be implemented by means of hardware comprising several different elements and by means of a properly programmed computer. In unit claims that list several devices, some of those devices may be embodied by the same item of hardware. The words first, second, third and so on do not denote any order. Those words may be interpreted as names.

Finally, it should be noted that the above embodiments are merely intended to explain the technical solutions of the present disclosure, and not to limit them. Although the present disclosure is explained in detail with reference to the above embodiments, a person skilled in the art should understand that he can still modify the technical solutions set forth by the above embodiments, or make equivalent substitutions to part of the technical features of them. However, those modifications or substitutions do not make the essence of the corresponding technical solutions depart from the spirit and scope of the technical solutions of the embodiments of the present disclosme.

1. A method for processing sleeping data, wherein the method comprises:
acquiring sleeping data collected by a sleeping monitoring device;
extracting a sleeping feature in the sleeping data;
performing similarity comparison to a standard feature of a user and the sleeping feature, to obtain a comprehensive feature similarity; and
on the condition that the comprehensive feature similarity satisfies a similarity requirement, using the sleeping feature as a target sleeping feature of the user.

2. The method according to claim 1, wherein the step of extracting the sleeping feature in the sleeping data comprises:
by using sleeping algorithms corresponding to sleeping sub-stages, acquiring sleeping datasets individually matching with the sleeping sub-stages in the sleeping data, and a sleeping-period time sequence of the sleeping data; and
according to the sleeping-period time sequence and the sleeping datasets, acquiring the sleeping feature.

3. The method according to claim 2, wherein the sleeping feature comprises at least: a respiration feature and a heartbeat feature; and
the step of performing the similarity comparison to the standard feature of the user and the sleeping feature, to obtain the comprehensive feature similarity comprises:
according to the sleeping-period time sequence, dividing the respiration feature and the heartbeat feature, to obtain sub-stage-feature sets corresponding to the sleeping sub-stages;
comparing each of the sub-stage-feature sets with the standard feature, to obtain feature similarities corresponding to the sleeping sub-stages; and
by using weight values corresponding to the sleeping sub-stages, integrating the feature similarities, to obtain the comprehensive feature similarity.

4. The method according to claim 1, wherein before the step of extracting the sleeping feature in the sleeping data, the method further comprises:
filtering data from the sleeping data that satisfy an ineffective-data requirement, wherein the ineffective-data requirement comprises at least one of an ineffective-data-format requirement and an ineffective-data-valuing requirement.

5. The method according to claim 2, wherein the sleeping feature comprises at least: a sleeping quality; and
the step of, according to the sleeping-period time sequence and the sleeping datasets, acquiring the sleeping feature comprises:
according to the sleeping datasets and the sleeping-period time sequence, acquiring an apnea-hypopnea index, an awakening time quantity, a falling-asleep duration, a sleeping duration and a sleeping efficiency; and
integrating the apnea-hypopnea index, the awakening time quantity, the falling-asleep duration, the sleeping duration and the sleeping efficiency, to obtain the sleeping quality.

6. The method according to claim 1, wherein the step of acquiring the sleeping data collected by the sleeping monitoring device comprises:
receiving heartbeat messages periodically reported by the sleeping monitoring device;
extracting a device state in the heartbeat messages;
on the condition that the device state is an operating state, sending a data acquiring request to the sleeping monitoring device; and
receiving the sleeping data that are sent by the sleeping monitoring device according to the data acquiring request.

7. The method according to claim 6, wherein before the step of receiving the heartbeat messages periodically reported by the sleeping monitoring device, the method further comprises:
acquiring a current time from a time calibrating server, to perform clock synchronization with the sleeping monitoring device.

8. The method according to claim 1, wherein after the step of using the sleeping feature as the target sleeping feature of the user, the method further comprises:
extracting, from a sleeping-suggestion information base, a target sleeping-suggestion information that matches with the target sleeping feature and a user information, and according to the target sleeping feature, generating a sleeping view; and
sending to a client a sleeping report that is formed by the sleeping view and the target sleeping-suggestion information, so that the client exhibits the sleeping report.

9. The method according to claim 8, wherein before the step of sending to the client the sleeping report that is formed by the sleeping view and the target sleeping-suggestion information, the method further comprises:
combining the sleeping view in a preset time period with the target sleeping-suggestion information, to obtain the sleeping report corresponding to the preset time period.

10. The method according to claim 9, wherein the step of combining the sleeping view in the preset time period with the target sleeping-suggestion information, to obtain the sleeping report corresponding to the preset time period comprises:
according to an operation parameter of the sleeping monitoring device, generating an operation-indicator information; and
combining the sleeping view in the preset time period, the target sleeping-suggestion information and the operation-indicator information, to obtain the sleeping report corresponding to the preset time period.

11. The method according to claim 8, wherein the step of extracting, from the sleeping-suggestion information base, the target sleeping-suggestion information that matches with the target sleeping feature and the user information comprises:
extracting, from the sleeping-suggestion information base, a sleeping-suggestion information that matches with the target sleeping feature and the user information; and
extracting, from the sleeping-suggestion information, the target sleeping-suggestion information that satisfies a user-configuration type, wherein the user-configuration type comprises at least one of an audio type, a video type and a text type.

12. (canceled)

13. A computing and processing device, wherein the computing and processing device comprises:
a memory storing a computer-readable code; and
one or more processors, wherein when the computer-readable code is executed by the one or more processors, the computing and processing device implements the operations comprise:
acquiring sleeping data collected by a monitoring device;
extracting a sleeping feature in the sleeping data;
performing similarity comparison to a standard feature of a user and the sleeping feature, to obtain a comprehensive feature similarity; and
on the condition that the comprehensive feature similarity satisfies a similarity requirement, using the sleeping feature as a target sleeping feature of the user.

14. A computer program, wherein the computer program comprises a computer-readable code, and when the computer-readable code is executed in a computing and processing device, the computer-readable code causes the computing and processing device to implement the method for processing sleeping data according to claim 1.

15. A non-volitile computer-readable medium, wherein the computer-readable medium stores a computer program of the method for processing sleeping data according to claim 1.

16. The computing and processing device according to claim 13, wherein the operation of extracting the sleeping feature in the sleeping data comprises:
by using sleeping algorithms corresponding to sleeping sub-stages, acquiring sleeping datasets individually matching with the sleeping sub-stages in the sleeping data, and a sleeping-period time sequence of the sleeping data; and
according to the sleeping-period time sequence and the sleeping datasets, acquiring the sleeping feature.

17. The computing and processing device according to claim 13, wherein the sleeping feature comprises at least: a respiration feature and a heartbeat feature; and
the operation of performing the similarity comparison to the standard feature of the user and the sleeping feature, to obtain the comprehensive feature similarity comprises:
according to the sleeping-period time sequence, dividing the respiration feature and the heartbeat feature, to obtain sub-stage-feature sets corresponding to the sleeping sub-stages;
comparing each of the sub-stage-feature sets with the standard feature, to obtain feature similarities corresponding to the sleeping sub-stages; and
by using weight values corresponding to the sleeping sub-stages, integrating the feature similarities, to obtain the comprehensive feature similarity.

18. The computing and processing device according to claim 13, wherein before the operation of extracting the sleeping feature in the sleeping data, the operations further comprise:
filtering data from the sleeping data that satisfy an ineffective-data requirement, wherein the ineffective-data requirement comprises at least one of an ineffective-data-format requirement and an ineffective-data-valuing requirement.

19. The computing and processing device according to claim 16, wherein the sleeping feature comprises at least: a sleeping quality; and
the operation of, according to the sleeping-period time sequence and the sleeping datasets, acquiring the sleeping feature comprises:
according to the sleeping datasets and the sleeping-period time sequence, acquiring an apnea-hypopnea index, an awakening time quantity, a falling-asleep duration, a sleeping duration and a sleeping efficiency; and
integrating the apnea-hypopnea index, the awakening time quantity, the falling-asleep duration, the sleeping duration and the sleeping efficiency, to obtain the sleeping quality.

20. The computing and processing device according to claim 13, wherein the operation of acquiring the sleeping data collected by the sleeping monitoring device comprises:
receiving heartbeat messages periodically reported by the sleeping monitoring device;
extracting a device state in the heartbeat messages;
on the condition that the device state is an operating state, sending a data acquiring request to the sleeping monitoring device; and
receiving the sleeping data that are sent by the sleeping monitoring device according to the data acquiring request.

21. The computing and processing device according to claim 13, wherein after the operation of using the sleeping feature as the target sleeping feature of the user, the operations further comprise:
extracting, from a sleeping-suggestion information base, a target sleeping-suggestion information that matches with the target sleeping feature and a user information, and according to the target sleeping feature, generating a sleeping view; and
sending to a client a sleeping report that is formed by the sleeping view and the target sleeping-suggestion information, so that the client exhibits the sleeping report.
